(19) United States
(12) Patent Application Publication
(10) Pub. No.: US 2021/0034801 A1
(43) Pub. Date: Feb. 4, 2021

(54) METHODS AND SYSTEMS FOR DESIGNING METAMATERIALS

(71) Applicant: Thornton Tomasetti, Inc., New York, NY (US)

(72) Inventors: Jeffrey Cipolla, Washington, DC (US); Corbin Robeck, Washington, DC (US); Abilash Nair, New York, NY (US)

(73) Assignee: Thornton Tomasetti, Inc., New York, NY (US)

(21) Appl. No.: 16/942,512

(22) Filed: Jul. 29, 2020

Related U.S. Application Data
(60) Provisional application No. 62/880,078, filed on Jul. 29, 2019.

(51) Int. Cl. G06F 30/23; G06F 17/16

(52) U.S. Cl. CPC G06F 30/23; G06F 2111/10; G06F 17/16

(57) ABSTRACT

Systems and methods for computing linear and non-linear explicit, matrix-free, statics with applications to functionally graded mechanical metamaterials. In some aspects, these systems and methods use an algorithm based on a special finite element formulation called the Jacobian Free Newton Krylov (JFNK) method.

### CROSS-REFERENCE TO RELATED APPLICATION

This application claims the benefit of U.S. Provisional Application No. 62/880,078, filed on Jul. 29, 2019, the contents of which is incorporated herein by reference in its entirety.

### TECHNICAL FIELD

The present disclosure relates to systems and methods for computing linear and non-linear explicit, matrix-free, statics with applications to functionally graded mechanical metamaterials.

### BACKGROUND

Metamaterials have attracted an explosion of interest recently because of their novel properties and intriguing applications across a range of fields. For a metamaterial structure to be effective it must have very specific waveguide properties dictated by an underlying theory or equation. To achieve the required material properties for the entire microstructure, each unit cell within the microstructure must be homogenized to ensure the cell's parameter combinations yield the correct elastic stiffness tensor and density (Gokhale et al. 2012). Once the cloak microstructure has been designed, it is then often desirable to evaluate the microstructure's time domain performance under a hydrostatic preload. The assembled microstructure must also often be evaluated under a variety of solutions cases (i.e., static, quasi-static, transient). If the analyses are large, computational cost can become an issue however more importantly it is not uncommon for materials models to only be applicable to a specific solution type (e.g., explicit transient dynamics). Therefore, if exotic material models are used, complications can arise finding analogous material models across all solution cases.

### BRIEF SUMMARY

The present disclosure provides various computer-implemented methods and systems for reducing the computational burden, in terms of time and resources, when modeling of problems involving shell finite elements. In some aspects, the disclosed methods and systems reduce or eliminate the above-identified problems in the art. In addition, selected aspects of the disclosure provide other benefits and solutions, as discussed in detail below.

In a first exemplary aspect, the disclosure provides a computer-implemented method for reducing the computational burden, in terms of time and resources, when modeling of problems involving shell finite elements, the method comprising: a) a pre-processing phase, wherein a mesh is generated by a processor, and problem data are specified; b) a solution phase comprising the steps of: i) deriving element equations; ii) deriving global-system matrix-free shell finite element equations, by defining elements that include: bending and membrane stiffness degrees of freedom; iii) evaluating coefficients in said element equations using the derived element equations and the derived global system matrix free shell finite elements equations; iv) adding load and boundary conditions to said elemental equations; and v) solving said elemental equations in place of a global system of equations; and c) a post-processing phase wherein computed data is displayed to a user interface of a display device.

In some aspects, said step for deriving said global system matrix-free shell finite elements equations further extends to the applications: a) static homogenization, b) acoustic cloak design, c) hydrostatic loading, d) long-duration dynamics using implicit integration, or e) vibroacoustics in the frequency domain. In some aspects, this step may further comprise one or more of the following steps: a) defining a membrane elemental stiffness matrix; b) defining a bending elemental stiffness matrix; c) defining a preconditioning scheme for displacement degrees of freedom; d) defining a preconditioning scheme for rotational degrees of freedom; e) deriving associated algorithms and functions associated with said global-system matrix-free shell finite elements; or f) computing solution to said global-system matrix-free shell finite element equations.

In some aspects, said global system matrix-free step includes one or more of the additional steps: selecting an iterative Krylov scheme; defining approximate restart of the iterative scheme using a Taylor series expansion to avoid a global system of equations; finding the increment based on the tangent stiffness; expressing the next increment using only the action of the internal forces; and using the selected Krylov scheme to solve for the next increment.

In some aspects, said preconditioning scheme is defined as the diagonal array elements of the shell element stiffness matrices for bending and membrane, respectively. In some aspects, the preconditioning scheme is defined using one or more unassembled shell element stiffness matrices.

In a second exemplary aspect, the disclosure provides a computer-implemented system for performing one or more of the methods described herein, or any subset of the steps associated with such methods. For example, in one aspect, the disclosure provides a system for reducing the computational burden, in terms of time and resources, when modeling a problem involving shell finite elements, comprising a processor configured to generate a mesh and receive user-specified problem data; derive one or more element equations; derive one or more global system matrix-free shell finite element equations, wherein said deriving step comprises defining elements that include i) bending and ii) membrane stiffness degrees of freedom; execute calls to one or more internal force routines from an explicit dynamics element and material library to incorporate material models; evaluate coefficients in said element equations using the derived element equations and the derived global system matrix-free shell finite element equations; add load and boundary conditions to said element equations; and solve said element equations.

This simplified summary of exemplary aspects of the disclosure serves to provide a basic understanding of the invention. This summary is not an extensive overview of all contemplated aspects, and is intended to neither identify key or critical elements of all aspects nor delineate the scope of any or all aspects of the invention. Its sole purpose is to present one or more aspects in a simplified form as a prelude to the more detailed description of the invention that follows. To the accomplishment of the foregoing, the one or more aspects of the invention include the features described and particularly pointed out in the claims.

### BRIEF DESCRIPTION OF THE DRAWINGS

FIG. 1 is a diagram illustrating homogenization of a unit cell in the context of acoustic cloaking.

FIG. 2 is a constrained elastic block subjected to uniform extension.

FIG. 3 is a deformed shape of uniform extension, compression, and shear of elastic block.

FIGS. 4A, 4B and 4C depict a set of graphs showing center line stress (σ_{yy} for extension/compression and τ_{xy} for shear).

FIG. 5A illustrates a simply supported shell plate subjected to uniform loading (Contour σ_{yy}).

FIG. 5B is a graph showing vertical displacement of shell plate under uniform transverse loading.

FIG. 6A illustrates an exemplary metamaterial unit cell.

FIG. 6B illustrates a deformed shape of horizontal extension homogenization of the unit cell.

FIG. 6C illustrates a deformed shape of vertical extension homogenization of the unit cell.

FIG. 6D illustrates a deformed shape of pure shear homogenization of the unit cell.

FIG. 7A illustrates a functionally graded metamaterial structure subjected to hydrostatic loading.

FIG. 7B are examples of uniformly graded (left) and functionally graded (right) periodic metamaterial structures.

FIG. 7C is an example of compression of a sheet of unit cells to test the homogenization assumptions. Left: deformed (scaled) and undeformed configurations. Right: displacement contour overlaid on the undeformed configuration.

FIG. 8 is a flowchart illustrating an exemplary method according to the disclosure.

### DETAILED DESCRIPTION OF EXEMPLARY ASPECTS

Exemplary aspects of the disclosure are described herein in the context of systems and methods for computing linear and non-linear explicit, matrix-free, statics with applications to functionally graded mechanical metamaterials. In some aspects, such systems and methods use an algorithm based on a special finite element formulation called the Jacobian Free Newton Krylov (JFNK) method. Those of ordinary skill in the art will realize that the following description is illustrative only and is not intended to be in any way limiting. Other aspects will readily suggest themselves to those skilled in the art having the benefit of this disclosure. Reference will now be made in detail to implementations of the example aspects, as illustrated in the accompanying drawings. The same reference indicators will be used to the extent possible throughout the drawings and the following description to refer to the same or like items.

Static Homogenization

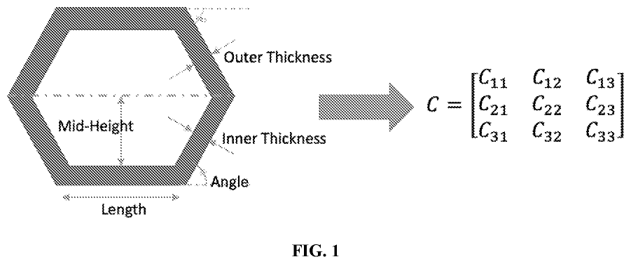

An inverse static homogenization process is required for the generation of a periodic metamaterial structure (Gokhale et al. 2012). A set of desired homogenized properties are prescribed, and a unit cell must then be found matching those homogenized properties (see, e.g., FIG. 1). As this generally yields non-unique solutions (i.e., more than one unit cell can match a set of homogenized properties), the inverse problem often requires searching a large, potentially non-linear, domain space of unit cells for desirable parameter combinations (Robeck et al. 2017). Moreover, the process of determining the material tensor of interest for a specific set of metamaterial cell design parameters involves solving a costly high dimensional finite element problem for each new microstructure cell of interest (Hassani and Hinton 1998). It is not uncommon for databases of homogenized unit cells to be in the high hundreds of thousands to millions. In practice, these high dimensional finite element problems are almost always solved using a direct or iterative solution of large system matrices to find a static solution (i.e., Ansys, Abaqus, LS-Dyna) for which homogenized properties can be determined. Even using advanced solutions methods, this approach can put significant demands on computational memory and processing power (Abaqus 2015). Furthermore, each unit cell solution can often fall in the “no man's land” of being too large to fit in a single CPU's available memory but generally still too small to take full advantage of distributed memory solution architectures (Robeck et al. 2017). Thus, deciding on how to make full use of available computational resources within the microstructure design can be a challenge. Allocating many single core simulations could be more attractive, from a throughput perspective, than using domain decomposition for each unit cell. Therefore, increasing single core solution efficiency is potentially quite valuable in the design process.

Quasi-static Loading

It is also often required to initialize quasi-static loads, such as preloads, on metamaterial microstructures either for direct analysis or for prior to its analysis in that the time. This hydrostatic preload allows a more accurate understanding of how the microstructures will behave in the intended use environment. A technique that is commonly done to establish a preload is dynamic relaxation (LS-Dyna 2007). Dynamic relaxation is a transient solution that relies on damping of the structure to achieve a quasi-static solution. Dynamic relaxation is able to take advantage of the explicit code's matrix free architecture, but is time intensive at achieving static equilibrium (Cassell and Hobb 1976). One particularly attractive aspect of dynamic relaxation is the prospect of initializing the preload directly in the explicit code (i.e., where the transient solution case will take place). There is then no need to undertake the somewhat cumbersome process of moving the solution data (i.e., displacement, stress, etc.) from static solution code to the explicit dynamics one. Thus, a preload solution procedure that can be integrated directly into the explicit dynamics loop has substantial value.

Selected Advantages of Matrix-Free Codes

Matrix free, explicit finite element codes are highly efficient, and widely used, for transient dynamic simulations in structures (i.e., LS-Dyna, Abaqus Explicit, etc.). Their efficiency stems from the use of central difference time-stepping which, when lumped inertia is used, results in decoupled (explicit) expressions for the solution increments. Only the internal force array needs to be evaluated at each increment, in contrast to implicit (matrix based) schemes where a tangent matrix (or Jacobian) must be evaluated and solved. The consequence of this decoupling is the ability to avoid construction of the large sparse system matrices ([K] and [M] for a structural dynamics problem) allowing explicit codes to solve much higher dimensional problems than are typically solved in implicit solution cases (i.e., modal analysis, acoustics, statics, etc.) due to the decreased memory demands in the explicit computation (Abaqus 2015). Therefore, a procedure in which the statics solution could be done entirely within the explicit dynamics framework, without having to resorting to time intensive dynamic relaxation, has attractive properties.

Jacobian-Free Methods

Recently, a specific family of Newton-type iterative solvers has been developed which avoid the construction and solution of the tangent or Jacobian matrix entirely (Knoll and Keyes 2003). These “Jacobian-Free Newton-Krylov” (JFNK) methods incorporate two main innovations. First, the effect of the Jacobian on a vector is approximated by a two-term Taylor expansion, avoiding the construction of the Jacobian matrix. Second, equilibrium at a Newton increment is found using a Krylov iterative solver, such as GMRES, CG, etc. In this manner, the JFNK methods allow explicit type solution approaches for problems which have typically been solved implicitly (i.e., full system level matrices). The entire acoustic metamaterials process: homogenization, hydrostatic preload, and time domain scattering analysis can then be done in the same code architecture, while potentially reducing time and memory demands substantially (Hales et al. 2012).

The JFNK method appears popular in computer science and large non-linear multi-physics problem classes (Geuzaine 2001, Rahul 2011, Hales et al. 2012, etc.) but its adoption in solid mechanics, especially for homogenization and metamaterials analysis, is relatively unexplored. Hales et al. (2012) investigated the method for the solution of large-scale multi-physics problems related to light water nuclear reactors. In their analysis, they implemented the method for non-linear springs and solids. They did not, however, address single integration point elements (and associated hourglass control)—which can have substantial cost savings over fully integrated elements, hyperelastic materials (which are becoming more common in metamaterials applications), or solution procedures for shells. Shells pose a particularly interesting problem as they are often necessary for a full mechanical metamaterial microstructure analysis. Furthermore, the method's applicability to the field of metamaterial homogenization and analysis is yet to be explored. Lastly, the process of converting an existing explicit dynamics finite element code to use JFNK for linear and/or non-linear statics appears entirely undocumented.

In selected aspects of the methods and systems described herein, the preconditioned conjugate gradient algorithm is coupled with the explicit JFNK procedure to solve the finite element based equations for linear and non-linear static equilibrium. Full-scale finite element examples are chosen to show the method's comparison to traditional implicit (matrix based) methods as well as its ability to handle a range of standard element types (solids and shells with hourglass control) and problem setups and scales. Strategies for preconditioning the system when no system level matrix exists (as is the nature of explicit methods) are described as well as JFNK's application to corresponding problems in mechanical metamaterial applications (acoustic cloaking in this case). The JFNK Conjugate Gradient (CG) is compared to its fully matrix-based counter-part as well as dynamic relaxation in an explicit dynamics code. The present disclosure further describes a process for integrating the JFNK solution procedure into an existing explicit dynamics element loop.

Equations of Motion

Following Newton's second law, the trajectory of any material point in a deformable body can be shown to follow:

$$ρ \frac{d v}{d t} = ∇ \cdot σ + b \qquad ( 1 )$$

where, v is the velocity of material point, σ is the Cauchy stress tensor, b is the body force acting in the current configuration of the body, and ρ is the material density. The operators V̅ and d/dt denote the directional gradient (with respect to spatial coordinates) and the material time derivative, respectively.

Finite Element Discretization

The discrete equations of motion can be derived from the strong form Eq. (1) using a variational approach (see Hughes 1981, Reddy 1984, and Belytschko et al. 2000 for an in-depth derivation). When inertia is neglected, we arrive at the linear system of equations usually represented in matrix form as,

Ku=F^{ext} (2)

where, K is a sparse matrix operator, u is the nodal displacement, and F^{ext} is a vector of external forces acting on the body. It is also recognized that Ku is the internal force vector F^{int}. From Eq. (2), the unknown displacements can be found through either direct or iterative methods to satisfy:

u=K⁻¹F^{ext} (3)

This equates to finding the displacement field u such that:

F^{int}=F^{ext} (4)

After the unknown displacements u are found through Eq. (3). the stress and strain can be found through:

ε=½[V̅u+(V̅u)^{T}] (5)

σ=C:ε (6)

where ε is the infinitesimal strain, C is the elastic stiffness tensor, and σ is the Cauchy stress.

Eq. (2) is a system of linear equations (Ax=b) and as such can be solved in a variety of ways. In this study, the well-known preconditioned conjugate gradient (CG) procedure is employed (Fletcher and Reeves 1964). CG is well studied to be robust but, like all iterative methods, the major drawback of CG, and indeed all methods that require a system level matrix, is the storage requirements to both assemble and solve the linear system. Even with ever growing computational power, modern problem sizes can easily overrun a machine's available memory. Multi-core, distributed memory, solution architectures seem like the obvious solution. However, in the case of unit cell homogenization, there are potentially millions of unit cell cases (Robeck et al. 2017) which can be too large for a single core's memory but not large enough to take advantage of a full message passing architecture (Geers et al. 2017). Additionally, since the homogenization process often requires multiple load cases to fully characterize the structure of interest, the prospect of removing an additional complexity (domain decomposition) and the need for a supercomputing cluster solely for the homogenization process from the process is an attractive offer.

Finite Deformation Statics

In the case of material non-linearity and/or large deformations, the equations must be solved iteratively (Belytschko et al. 2000). From Eq. (2), Ku can be combined into the internal force vector and the solution can be found by the minimization of:

R=F^{int}−F^{ext} (7)

where R is the residual force vector, F^{int} is the internal force vector, and F^{ext} is the external force acting on the body.

Equation Eq. (7) is iterated on until R is driven below a specified tolerance. A popular numerical scheme for non-linear statics is Newton-Raphson (NR) Iterations (Abaqus 2015, Ansys 2018, etc.). The procedure for an iterative NR solution is as follows:

Solving Eq. (7), we expand Ru in a Taylor series about a known iterate u^{k}, such that

R(u^{k}+Δu)=R(u^{k})+K_{T}:Δu+O(Δu²) (8)

where

$${K}_{T} = \frac{\partial {F}^{i n t}}{\partial u}$$

is the tangent matrix. Retaining only first order terms we come to,

K_{T}:Δu=−R(uₖ) (9)

u^{k+1}=u^{k}+Δu (10)

However, because the strain-displacement relationship is now non-linear, Eq. (9) must be solved at every iteration. It is noted that the iterations require the formation of the tangent stiffness, K_{T}, for the solution of the non-linear problem Eq. (9), as well as the solution of the linear system Eq. (2) at each increment (Belytschko et al. 2000). Stated another way, both outer iterations (referred to as load steps) and inner iterations are required in the non-linear solution loop which can become computationally expensive depending on the application.

Large Deformation Statics—Hyperelasticity

Metamaterials applications involving non-linear materials have become more common (e.g., polymers, rubber, etc.) in recent years (Florijn et al. 2014, Rafsanjani 2015, etc.). These types of materials display force-displacement non-linearity and the potential for large deformations. Therefore, a corresponding constitutive model must be chosen that accurately relates stress to strain. For this study, non-linear materials were investigated through hyperelasticity as they have begun to appear more often in metamaterials applications. Within the finite strain solution, the calculation of R remains the same as Eq. (7), however a Neo-Hookean (Ogden 1984) approach for hyperelasticity is followed such that the stress is

$$\begin{matrix}σ = \frac{G}{{𝒥}^{\frac{5}{3}}} ( B - \frac{1}{3} t r ( B ) I + K ( 𝒥 - 1 ) I & ( 11 ) \\ B = {F}^{T} F & ( 12 ) \\ 𝒥 = det ( F ) & ( 13 )\end{matrix}$$

where [symbol] is the Jacobian determinant, G is the shear modulus, K is the bulk modulus, B is the left Cauchy-Green strain tensor, F is the deformation gradient, and I is the identity matrix.

Even in this relatively simple non-linear constitutive model, the material tangent stiffness calculation is tedious to unravel (Ansys 2018),

$${K}_{T} = \frac{2}{3} G {𝒥}^{- \frac{2}{3}} [ - {C}^{- 1} ⊗ I - I ⊗ {C}^{- 1} + tr ( C ) {C}_{i k}^{- 1} {C}_{j l}^{- 1} + \frac{tr ( C )}{3} {C}^{- 1} ⊗ {C}^{- 1} ] + K 𝒥 [ ( 2 𝒥 - 1 ) {C}^{- 1} ⊗ {C}^{- 1} - 2 ( 𝒥 - 1 ) {C}_{i k}^{- 1} {C}_{j l}^{- 1} ] \qquad ( 14 )$$

where K_{T} is the tangent stiffness matrix, [symbol] is the Jacobian determinant, G is the shear modulus, K is the bulk modulus, C is the Cauchy-Green deformation tensor, and I is the identity matrix.

Therefore, a solution procedure in which K_{T} is not required in closed form could prove valuable should more exotic constitutive be explored as interest in metamaterials develops.

Matrix-Free Methods

Conjugate Gradient Jacobian Free Newton-Krylov (CG-JFNK)

To investigate matrix-free solutions to Eq. (7), the Jacobian Free Newton-Krylov (JFNK) procedure may be followed to solve the governing equation of motion, Eq. (2). A Taylor expansion of the problem is pursued (similar to above), but the tangent matrix is never explicitly formed. These iterations solve the same equation as Eq. (9) however using,

K_{T}:s=−R(uₖ) (15)

where, s=u*−u. The left hand-side of the above equation is approximated using the following finite difference formula,

$${K}_{T} ( u ) \text{:} s \approx {K}_{T} ( s , u ) = \frac{R ( u + h s ) - R ( u )}{h} \qquad ( 16 )$$

This is the critical step which avoids the formation of K_{T} and enables the JFNK process to be executed using only calls to internal force routines, as in explicit dynamics. In this work, Eq. (16) is solved for s using the CG method as well. The solution n+1 is determined from:

uⁿ⁺¹=uⁿ+s (17)

It is seen that the solution is dependent on the value chosen for the parameter “h”. In the following results, h is determined using the formula,

$$h = \sqrt{\frac{( 1 + ‖ u ‖ ) ϵ}{‖ s ‖}} \qquad ( 18 )$$

where ϵ is the machine epsilon.

An important feature of JFNK for solid mechanics is that the procedure is inherently non-linear in its formulation therefore in structural mechanics with non-linear materials the tangent stiffness need not be known in advance (or even exist). In the case of non-linear materials with complicated strain-displacement relationships, it may be quite attractive to not be required to compute this tangent stiffness directly. For JNFK, the only requirement is the action of the tangent stiffness operator on the incremental displacement field—which could be considerably easier to compute numerically (Hales et al. 2016). In the case of the Neo-Hookean material described above in the context of large deformation statics, tangent stiffness matrix calculation can be replaced by Eq. (16) which can have considerably fewer operations. Additionally, while the tangent stiffness equation exists for Neo-Hookean materials, albeit somewhat cumbersome in nature, Hales et al. (2016) points out there are many cases of non-linear materials where no analytical description of the tangent stiffness matrix exists at all making JFNK an attractive option. Lastly, and perhaps the most appealing aspect of JFNK in the context of homogenization is the ability to run large degree of freedom problem sets without constraints on memory requirements since no system level matrix assembly is required. In explicit dynamics, the computational cost is a function of the number of elements and the smallest element characteristic length (Abaqus 2015). In JFNK. however, since there is no time stepping, the dependency on the characteristic length is eliminated entirely.

Pre-Conditioned JFNK

The JFNK solution procedure is well-known to improve through use of a preconditioner. However, in the Jacobian-Free context, no system level matrix exists making assembling a preconditioning matrix less straight forward than typical iterative schemes (Knoll and Keyes 2004, Hales et al. 2012, and others). Hales et al. (2012) and Knoll and Keyes (2003) discuss a variety of preconditioning options; however, Hales et al. notes that solution time and even the ability to find a solution at all for a particular problem class is highly dependent on the preconditioner chosen. Additionally, since one of JFNK's appealing features is its ability to seamlessly integrate into an existing explicit element loop, any precondition technique that requires operations to occur outside of the internal force calculation is unattractive. Therefore, we use a segmented diagonal preconditioning scheme that can be calculated entirely on the elemental level with no interaction of system level matrices (or even data structures outside of the element procedures). Following the right-hand pre-conditioning of Eq. (16) we arrive at

$${K}_{T} ( u ) {P}^{- 1} \text{:} Ps \approx {K}_{T}^{h} ( {P}^{- 1} \hat{s} , u ) = \frac{R ( u + h \hat{s} ) - R ( u )}{h} \qquad ( 19 )$$

where we have set, ŝ=Ps and P is the pre-conditioner. The converged displacement update is provided as:

uⁿ⁺¹=uⁿ+Pŝ (20)

Preconditioning is done during the explicit element loop such that:

P_{Element}=diag(K_{Element}) (21)

where P_{Element} is the diagonal preconditioning matrix of the element and K_{Element} is the elemental stiffness matrix. While more exotic efficient preconditioners exist, this preconditioning scheme was chosen because it converged consistently for all problem classes and elements studied.

Single Integration Point Methods

Single integration point elements are popular in explicit dynamics due their speed and decreased operation count (Belytschko et al. 2010). However single integration point elements require hourglass control to remedy spurious modes that are generated (see Flanagan and Belytschko et al. (1981) and Flanagan and Belytschko (1984) for a thorough discussion of hourglass control). In this study, perturbation based hourglass control is used to augment the element stiffness matrix to reduce element hourglassing in solid elements, while stabilization is used for shells, as discussed herein. For reduced integration elements, the effect of the hourglass forces on convergence was found to be minimal. This is not that surprising, however, as the artificial hourglass stiffness terms added to the element stiffness matrix are relatively small in relation to the inertial terms. The addition of the hourglass stiffness terms to the precondition scheme did however slightly increase preconditioning efficiency—although in no case was it found to be by more than a few iterations. Nevertheless, the hourglass stiffness terms were added to the preconditioning matrix such that Eq. (21) was modified to be

P_{Element}=diag(K_{Element}+K_{HG}) (22)

where K_{HG} are the hourglass stiffness terms. Eq. (22) was used to precondition the system whenever reduced integration elements with hourglass control were used.

Conjugate Gradient JFNK (CG-JFNK) for Shells

Shells are tricky to implement in JFNK as they have multiple elemental stiffness matrices (bending and membrane) of often very varied magnitudes (Reddy 1984, Hughes 1987, and others). This makes preconditioning for shell elements more challenging than elements with more uniform element stiffness matrices (springs, solids, etc.). As such taking the preconditioning matrix as described in Eq. (21) above often results in non-convergence within a reasonable number of iterations. Therefore, a special shell preconditioning routine was implemented. The shell formulation used is a reduced integration 4 node, 24 degree of freedom (3 displacement and 3 rotation at each node), bilinear quad with decoupled membrane and bending stiffness components (i.e., first order shear deformation theory). Hourglass modes are controlled by the stabilization method proposed by Belytschko et al. (1981). A thorough discussion of the element's shape functions and strain displacement relationships is available in Averill and Reddy (1990) and Reddy (1999). Once the elemental stiffness matrix is assembled, the element internal force vector can then be found as,

F_{Element}^{int}=−K_{Element}u_{Element} (23)

where, Fₑₗₑₘₑₙₜ^{int} is the internal force vector of the shell element, K_{Element} is the elemental stiffness matrix, and u_{Element} is the displacement vector (displacement and rotations in the case of shells) of the element. Eq. (23) is noted as being a general relationship that exists regardless of element type (i.e., not unique to shells).

Preconditioning for shell elements follows a similar approach as for solid elements however the force (F) and moment (M) degrees of freedom are separated and preconditioned independently.

Let xₙ=(μₙ, θₙ)^{T} be the converged displacements (uₙ) and rotations (θₙ) from step “n”. We can then define the following quantities,

r₀^{F}=Fₙ^{int}−Fₙ₊₁^{ext} (24)

r₀^{M}=Fₙ^{int}−Fₙ₊₁^{ext} (25)

Also, set k=1, Δu=0, Δθ=0, and the following quantities

$$\begin{matrix}{ρ}_{0}^{F} = {r}_{0}^{F} \cdot {r}_{0}^{F} & ( 26 ) \\ {ρ}_{0}^{M} = {r}_{0}^{M} \cdot {r}_{0}^{M} & ( 27 ) \\ {p}_{1}^{F} = \frac{{r}_{0}^{F}}{\sqrt{{ρ}_{0}^{F}}} & ( 28 ) \\ {p}_{1}^{M} = \frac{{r}_{0}^{M}}{\sqrt{{ρ}_{0}^{M}}} & ( 29 )\end{matrix}$$

The action of the Jacobian through finite difference approximation can then be found as,

$$\begin{matrix}{k}_{F}^{} = {J}^{h} ( {p}_{k}^{F} , {p}_{k}^{M} ) Δ {u}_{n + 1} & ( 30 ) \\ {k}_{M}^{} = {J}^{h} ( {p}_{k}^{F} , {p}_{k}^{M} ) Δ {θ}_{n + 1} where , & ( 31 ) \\ {J}^{h} Δ {u}_{n + 1} \approx \frac{{F}^{int} ( {u}_{n} + h {p}_{k}^{F} , {θ}_{n} + h {p}_{k}^{M} ) - {F}^{int} ( {u}_{n} , {θ}_{n} )}{h} & ( 32 ) \\ {J}^{h} Δ {θ}_{n + 1} \approx \frac{{M}^{int} ( {u}_{n} + h {p}_{k}^{F} , {θ}_{n} + h {p}_{k}^{M} ) - {M}^{int} ( {u}_{n} , {θ}_{n} )}{h} & ( 33 )\end{matrix}$$

Update incremental displacements, rotations, and residuals using line search,

$$\begin{matrix}{α}_{k}^{F} = \frac{{ρ}_{k - 1}^{F}}{{p}_{k}^{F} \cdot {w}_{k}^{F}} & ( 34 ) \\ {α}_{k}^{M} = \frac{{ρ}_{k - 1}^{M}}{{p}_{k}^{M} \cdot {w}_{k}^{M}} & ( 35 ) \\ Δ {u}_{n + 1}^{k} = Δ {u}_{n + 1}^{k - 1} {α}_{k}^{F} {p}_{k}^{F} & ( 36 ) \\ Δ {θ}_{n + 1}^{k} = Δ {θ}_{n + 1}^{k - 1} {α}_{k}^{M} {p}_{k}^{M} & ( 37 ) \\ {r}_{k}^{F} = {r}_{k - 1}^{F} - {α}_{k}^{F} {w}_{k}^{F} & ( 38 ) \\ {r}_{k}^{M} = {r}_{k - 1}^{M} - {α}_{k}^{M} {w}_{k}^{M} & ( 39 )\end{matrix}$$

Evaluate stopping criterion,

Pₖ^{F}=rₖ^{F}·rₖ^{F} (40)

ρₖ^{M}=rₖ^{M}·rₖ^{M} (41)

If \sqrt{ρₖ^{F}}<δ_{F}\sqrt{ρ₀^{F}} and \sqrt{ρₖ^{M}}<δ_{F}\sqrt{ρ₀^{M}} then set,

xₙ₊₁=xₙ(Δuₙ₊₁^{k},Δθₙ₊₁^{k})^{T} (42)

and exit loop.

Update search directions,

$$\begin{matrix}{β}_{k}^{F} = \frac{{ρ}_{k}^{F}}{{ρ}_{k - 1}^{F}} & ( 43 ) \\ {β}_{k}^{M} = \frac{{ρ}_{k}^{M}}{{ρ}_{k - 1}^{M}} & ( 44 ) \\ {p}_{k + 1}^{F} = {r}_{k}^{F} + {β}_{k}^{F} {p}_{k}^{F} & ( 45 ) \\ {p}_{k + 1}^{M} = {r}_{k}^{M} + {β}_{k}^{M} {p}_{k}^{M} & ( 46 )\end{matrix}$$

The algorithm presented above is generalized to allow for an arbitrary number of “n” load steps. Load steps are required when inducting plasticity or material non-linearity.

Incorporating JFNK into Explicit Time-Integration Algorithms

Dynamic Relaxation is the incumbent method in explicit dynamics codes (e.g., Abaqus Explicit, LS-Dyna, etc.) to achieve a quasi-static solution. The steady state problem is converted to a pseudo-transient problem such that Eq. (7) now becomes,

R−C\dot{u}−Mü=0 (47)

Here, the “time” should be understood as artificial. In the DR method, C=cM and the matrix M is a lumped mass matrix (only diagonal terms exist). A central-difference method is adopted such that the velocity update at step “n” is,

$${\dot{u}}^{n + \frac{1}{2}} = \frac{2 Δ t}{2 + c Δ t} {M}^{- 1} {R}^{n} + ( \frac{2 - c Δ t}{2 + c Δ t} ) {\dot{u}}^{n - \frac{1}{2}} \qquad ( 48 )$$

At the first time-step the velocity is predicted to be,

$${\dot{u}}^{\frac{1}{2}} = \frac{( 2 - c Δ t )}{2} {\dot{u}}^{0} + \frac{Δ t}{2} {M}^{- 1} {R}^{0} \qquad ( 49 )$$

The displacement update is simply,

uⁿ⁺¹=uⁿ+Δt\dot{u}^{n+1/2} (50)

The time step for a given problem can be chosen such that the critical time-step criterion is always satisfied,

$$Δ t = \frac{2}{\sqrt{{λ}_{m}}} \qquad ( 51 )$$

where,

$${λ}_{m} = max {\sum }_{j = 1}^{n} \frac{\vert {( {K}_{T} )}_{ij} \vert}{{m}_{tj}} ,$$

with K_{T} being the tangent stiffness matrix.

The optimal damping coefficient is chosen such that,

$$\begin{matrix}c \leq 2 \sqrt{{λ}_{0}} & ( 52 ) \\ where , & \\ {λ}_{0} = \frac{Δ {u}^{T} ( {F}^{int , n + 1} - {F}^{int , n} )}{Δ {u}^{T} M Δ u} & ( 53 )\end{matrix}$$

with Δu=Δt\dot{u}^{n-1/2}.

The optimal time step solution procedure is referred to as “adaptive” dynamic relaxation (LS-Dyna 2007). Adaptive dynamic relaxation does, however, require the formation of the tangent stiffness matrix K_{T} in Eq. (50) in order to have optimal time-stepping (an operation not required in JFNK). Another additional required step, compared to both matrix-based statics and JNFK, is the need for the mass matrix, M. M is lumped (i.e., diagonal) and therefore does not place large demands on memory. It does, however, represent an additional computational step that is not required in JFNK or standard matrix-based statics.

Description of the Adaptive Dynamic Relaxation (aDR) Algorithm

Initialization for timestep n=0:
1. Require: u⁰=uⁿ
2. Calculate residual R⁰ and internal force vector F^{int,0}
3. Form tangent stiffness K_{T}(u⁰)
4. Determine critical timestep Δt from Eq. (51)
5. Update velocity using Eq. (49) with u⁰=0 and c=0
6. Set the iterate within each timestep k=1

Element loop:
1. Find displacement u^{k} using Eq. (50)
2. Find the internal force vector F^{int,k}, residual R^{k}, and tangent stiffness K_{T}(u^{k})
3. Determine the optimal time-step Δt from Eq. (51)
4. Find the damping coefficient c from Eq. (52) and Eq. (53)
5. Update velocities using Eq. (49)
6. If eₖ=MAX(∥\dot{u}^{k+1}∥², ∥R^{k+1}∥)<tolerance then
set uⁿ⁺¹=u^{k+1}
break loop
else set k=k+1
   for the purposes of this disclosure, a kinetic energy ratio (eₖ) tolerance for static state criterion was chosen to be 0.1E-04 erg (using cgs units).

Integration of JFNK into an Existing Explicit Dynamics Finite Element Code

As noted, JFNK is particularly attractive due to its ease of integration into an existing explicit dynamics code. The entire framework of the explicit dynamics code remains unchanged to incorporate JFNK, the only requirement are calls to the element force routine of Eq. (5).

Description of an Explicit Dynamics Routine

Initialization:
1. Determine the initial acceleration of element i from the residual force vector and diagonalized mass matrix:

$${a}_{i}^{0} = \frac{{R ( {u}_{i}^{0} )}_{i}^{0}}{{M}_{i}} \qquad ( 54 )$$

2. Find initial velocities from elemental accelerations and initial timestep:

$${v}_{i}^{0} = \frac{1}{2} Δ {ta}_{i}^{0} \qquad ( 55 )$$

3. Update initial displacements:

uᵢ⁰=Δtvᵢ⁰ (56)

where aᵢ⁰, vᵢ⁰, uᵢ⁰ are the ith element's initial acceleration, velocity, and displacement.

Time Stepping:

Loop over i elements for n time steps:
1. Determine elemental reactions force, Fᵢ^{int}(uᵢⁿ) as a function of the new displacement field:

Fᵢ^{int}=∫Bₙ^{T}σₙdVᵢ
2. Explicitly compute accelerations, velocities, and displacement using central difference:

$$\begin{matrix}{a}_{i}^{n} = \frac{1}{{M}_{i}} [ {F}^{ext} - {F}^{init} ( {u}_{i}^{n} ) ] & ( 57 ) \\ {v}_{i}^{n + \frac{1}{2}} = {v}_{i}^{n - \frac{1}{2}} + \frac{1}{2} ( Δ {t}^{n - \frac{1}{2}} + Δ {t}^{n + \frac{1}{2}} ) {a}_{i}^{n} & ( 58 ) \\ {u}_{i}^{n + 1} = {u}_{i}^{n} + Δ {t}^{n + \frac{1}{2}} {v}_{i}^{n + \frac{1}{2}} & ( 59 )\end{matrix}$$

As can be seen, there are no system level matrices anywhere in the algorithm. Computationally the most expensive part of the algorithm is evaluating the internal force routine for each element at each time step. However, because these calculations are done entirely on the element level, memory requirements are quite low (compared to matrix based methods).

Description of Explicit Preconditioned-Conjugate Gradient JFNK (PCG-JFNK)

The process of using an existing explicit dynamics loop to perform explicit (i.e., matrix free) statics with JFNK is straightforward. The algorithm uses the element force call from Eq. (18) directly from the explicit dynamics code with no modifications to either interface or internal routines.

Initialization:
1. s⁰=0
2. Assemble the external force vector acting on the body F^{ext}
3. Assemble internal force vector F^{int} from prescribed displacements
F^{int}(u⁰) and preconditioning vector P(u⁰).
4. Set r=−[F^{ext}−F^{int}(u⁰)] following Eq. (17).
5. Set z⁰=P⁻¹r⁰, recall P is diagonal.
6. Set v⁰=z⁰.

Element loop for i elements and n iterations:
1. Solve for the incremental displacement, u*

u*=uⁿ+hvⁿ
2. Find F^{int}(u*ᵢ)ᵢ and preconditioning vector P(u*ᵢ)ᵢ with elemental force call from Eq. (23).

$$\begin{matrix}Set {r}^{n} = {F}^{ext} + {F}^{int} . & 3 \\ {g}_{i} = ( [ {F}^{int} ( {u}_{i}^{*} ) - {F}^{ext} ] - [ {F}^{int} ( {u}_{i} ) - {F}^{ext} ] ) / h . & 4 \\ {α}_{i} = \frac{{z}_{i}^{T} \cdot {r}_{i}}{{v}_{i} \cdot {g}_{i}} . & 5 \\ {s}_{i + 1} = {s}_{i} + {α}_{i} {v}_{i} . & 6\end{matrix}$$

7. Evaluate stopping criterion. If residuals are sufficiently small then set,

uⁿ⁺¹=sᵢ₊₁
Exit solution loop, continue to next load step

$$\begin{matrix}{z}_{i + 1} = {P}^{- 1} {z}_{i} . & 8 \\ {β}_{i} = \frac{{z}_{i + 1} {r}_{i + 1}}{{z}_{i} {r}_{i}} . & 9 \\ {v}_{i + 1} = {z}_{i + 1} + {β}_{i} {v}_{i} . & 10 \\ set i = i + 1. & 11\end{matrix}$$

End Loop

As can be seen, like explicit dynamics, there are no system level matrices anywhere in the algorithm. The preconditioning matrix, P, is assembled and used entirely on the elemental level.

### EXAMPLES

### Example 1: Elastic Blocks

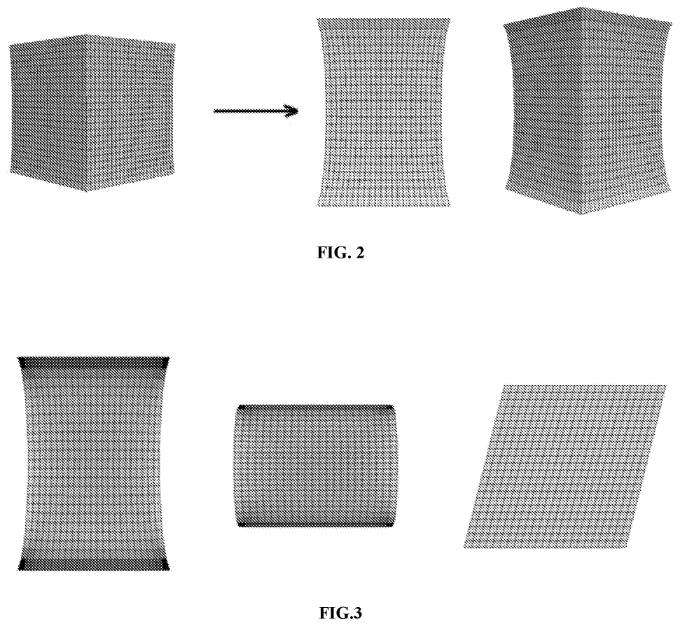

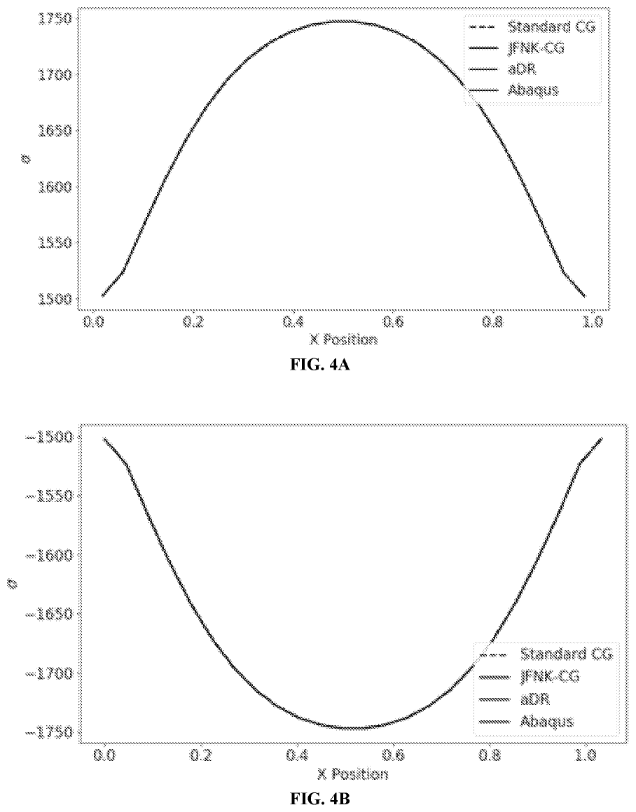

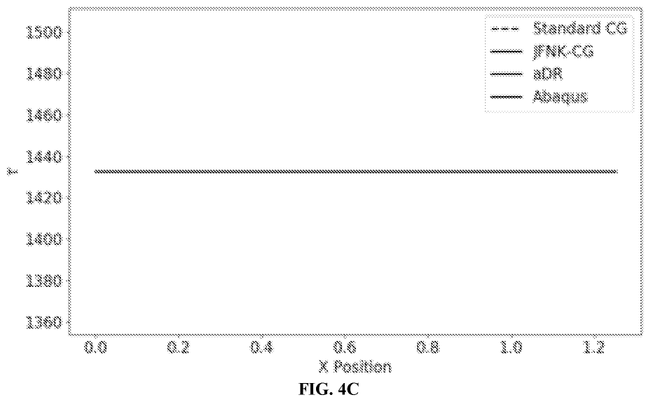

As illustrated by FIGS. 2 and 3, a 1.0 cm³ block was meshed with 12,167 eight-noded, fully-integrated hex elements (no hourglass control). The cube is deformed three separate times to evaluate tension, compression, and shear. A linear elastic material model was used with Young's modulus of 14,900 Pa, and Poisson's ratio of 0.3. FIG. 4 illustrate the center line stress (σ_{yy}, for extension/compression and τ_{xy} for shear) calculated in this example.

TABLE 1
Performance of Standard CG, JFNK CG, aDR,
and Abaqus for an exemplary elastic block.

| | | Required | |
|---|---|---|---|
| | Number | Solution | Extension |
| Solution | of | Memory | Peak |
| Method | Iterations | (MB) | σ_{yy} |
| Standard CG | 123 | 185.6 | 1747.06 |
| JFNK CG | 174 | 0.15 | 1747.06 |
| Adaptive | 125, 164 | 0.16 | 1751.32 |
| Dynamic | | | |
| Relaxation | | | |
| Abaqus | 11 | 248.1 | 1747.06 |
| Standard | | | |

The results shown above were obtained with algorithms prototyped in Python and tested in Fortran/C++ and compared to the Abaqus finite element analysis package. In JFNK-CG and explicit dynamic relaxation the only matrices that are assembled are on the element level. These matrices are the strain displacement matrix, B, the diagonal preconditioning matrix P_{Element}, and the elemental stiffness matrix, K_{Element}. For standard CG, the major memory usage is storing and solving the system level stiffness matrix [K]. [K] is constructed using Linked List Format (LIL) and converted to Compressed Sparse Row (CSR) for the matrix vector operations needed in CG.

To show JFNK's efficiency over traditional matrix based methods, the elastic block problem was scaled up to 210k elements (5.04 million DOFs). In this case, the problem solution requires more memory than available than on the CPU tested (2 GB). As expected, the matrix-based solution methods fail to complete. JFNK, however, because its memory efficient formulation, is able to find the solution. It is also noted that the vast majority of the memory requirements in the explicit methods are outside the actual solution procedure (e.g., storing node and element definitions). The actual solution loop memory requirements within explicit analysis remain relatively constant regardless of the number of DOFs in the problem (Hallquist 2012).

TABLE 2
Performance of Standard CG, JFNK CG, aDR,
and Abaqus for a large elastic block.

| | | Required |
|---|---|---|
| | Number | Solution |
| Solution | of | Memory |
| Method | Iterations | (MB) |
| Standard CG | Did Not Finish | 1968.2 |
| JFNK CG | 1579 | 115.1 |
| Adaptive | 171, 418 | 116.5 |
| Dynamic | | |
| Relaxation | | |
| Abaqus | Did Not Finish | 2485.1 |
| Standard | | |

Note:
Physical memory was limited to 2 GB to prevent swapping

It is well established that mesh refinement in three dimensions nominally increase memory requirements by a factor of 64 (2⁶) for matrix-based solutions while memory costs only increase by a factor of 8 in explicit (e.g., JNFK) analysis (Abaqus 2015). Therefore, JFNK should continue to scale well for still further refinement.

### Example 2: Shell Plate In Bending

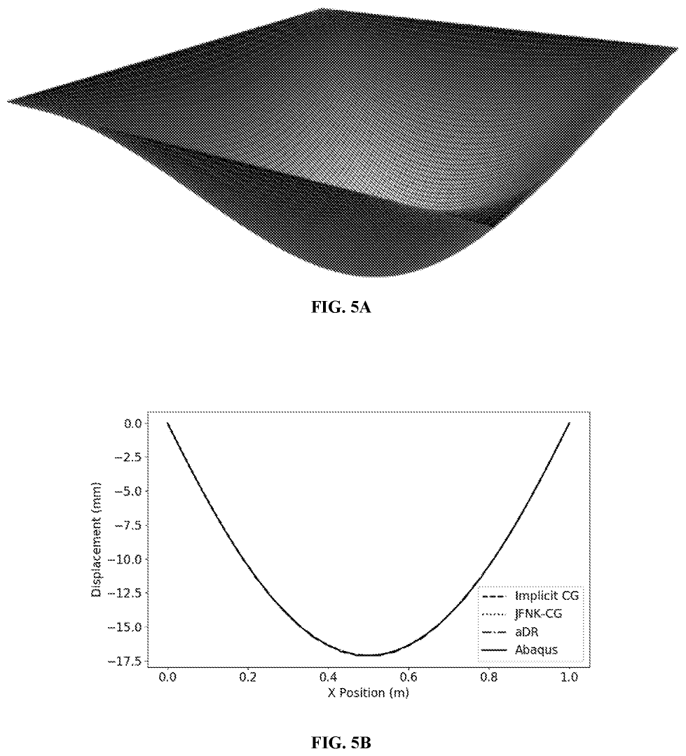

A 1.0 m², 1 mm thick, elastic plate under uniform transverse loading was analyzed to evaluate JFNK's performance with shells, as illustrated by FIG. 5A. A similar problem was investigated by Hales et al. (2012), however, without the use of shell elements. The plate was meshed with 10,000 four node, single integration point shell elements. A linear elastic material model was used with Young's modulus of 207 GPa, mass density of 7.83 g/cm3, and Poisson's ratio of 0.32. The vertical displacement of this exemplary shell plate under uniform transverse loading is depicted by the graph shown in FIG. 5B.

TABLE 3
Performance of Standard CG, JFNK CG, aDR,
and Abaqus for Shell Plate.

| | | Peak | |
|---|---|---|---|
| | Number | Memory | Peak |
| Solution | of | Usage | Deflection |
| Method | Iterations | (MB) | (mm) |
| Standard CG | 123 | 112.48 | 17.0 |
| JFNK CG | 147 | 0.22 | 17.0 |
| Adaptive | 151, 698 | 0.24 | 16.93 |
| Dynamic | | | |
| Relaxation | | | |
| Abaqus | 11 | 173.0 | 17.1 |
| Standard | | | |

### Example 3: Mechanical Metamaterial Unit Cell Homogenization

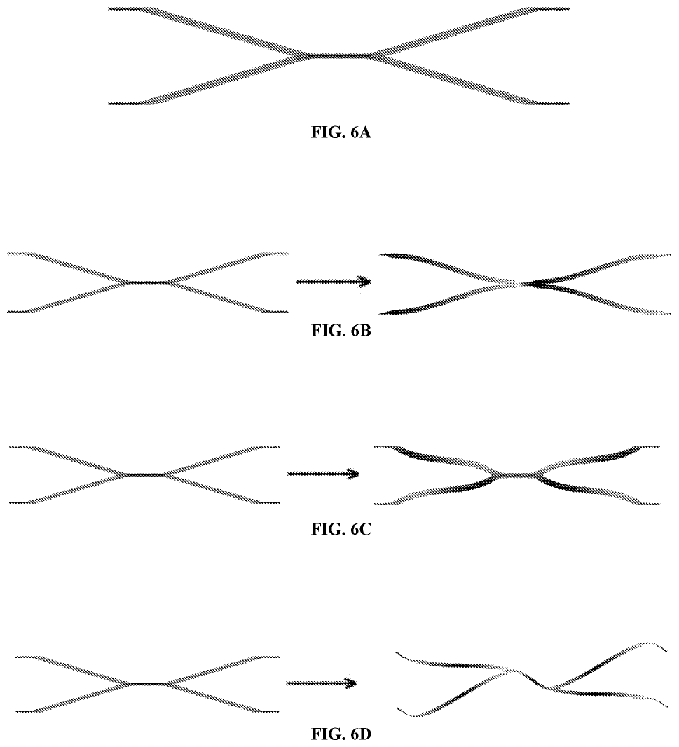

An elastic unit cell was homogenized to determine the elastic stiffness tensor. The unit cell was meshed with 2,616 four-node, single-integration-point, plane strain, quad elements with perturbation hourglass control (Flanagan and Belytschko 1984). A mass density of 7.83 g/cm³ and linear elastic material model with Young's modulus equal to 200 GPa and Poisson's ratio of 0.3 were used. Periodic boundary conditions were used on the left and right edges. For the case of plane strain, the generalized Hooke's law equation from Eq. (6) is replaced with

$$σ = \frac{E}{( 1 + v ) ( 1 - 2 v )} [ \begin{matrix}1 - v & v & 0 \\ v & 1 - v & 0 \\ 0 & 0 & 1 - 2 v\end{matrix} ] ɛ \qquad ( 60 )$$

where σ is the Cauchy stress tensor, E is the Young's modulus and v is the Poisson's ratio, and ε the infinitesimal strain. See FIG. 6A (depicting an acoustic cloak unit cell in accordance with this example).

Hassani and Hinton (1998) showed for 2D problems with periodic boundary conditions all of the elastic stiffness coefficients can be found through just three loading cases (vertical, horizontal, and shear) using a virtual displacement field. Therefore three separate strain fields were applied to predict effective constitutive properties and ultimately get to an effective elastic stiffness tensor for the unit cell:

$$[ {C}^{H} ] = [ \begin{matrix}{C}_{11}^{H} & {C}_{12}^{H} & 0 \\ {C}_{21}^{H} & {C}_{22}^{H} & 0 \\ 0 & 0 & {C}_{66}^{H}\end{matrix} ] \qquad ( 61 )$$

where C^{H} is the homogenized elastic stiffness tensor with equivalent properties of the unit cell and Cᵢⱼ^{H} are the homogenized elastic stiffness components.

In the context of 3D finite elements, the periodic homogenization equation can be written as a sum over N elements as

$${C}_{ijkl}^{H} = \frac{1}{Y} {\sum }_{e = 1}^{N} {( {u}_{e}^{ij} )}^{T} {k}_{e} ( {u}_{e}^{kl} ) \qquad ( 62 )$$

where Y is the volume of non-void material in the unit cell, uₑ are the generated elemental displacements, and kₑ is the element stiffness (Gao et al. 2018). For 2D, only two indices are needed in Eq. (62). The calculation of C₁₁, C₂₂, and C₁₂ are shown.

3.1 Horizontal Extension (C11)

From a horizontal unit displacement field (u¹¹), the C₁₁ element of the homogenized elasticity tensor can be solved by

$${C}_{11}^{H} = \frac{1}{Y} {\sum }_{e = 1}^{N} {( {u}_{e}^{11} )}^{T} {k}_{e} ( {u}_{e}^{11} ) \qquad ( 63 )$$

See FIG. 6B, which illustrates the deformed shape of horizontal extension homogenization of the unit cell.

TABLE 4
Performance of Standard CG, JFNK CG, aDR, and
Abaqus for Horizontal Extension Homogenization.

| | | Peak | |
|---|---|---|---|
| | Number | Memory | |
| Solution | of | Usage | Solution |
| Method | Iterations | (MB) | (C₁₁) |
| Standard CG | 36 | 52.3 | 14.93 GPa |
| JFNK CG | 41 | 0.08 | 14.93 GPa |
| Adaptive | 100, 145 | 0.1 | 15.13 GPa |
| Dynamic | | | |
| Relaxation | | | |
| Abaqus | 10 | 67.1 | 14.93 GPa |
| Standard | | | |

3.2 Vertical Extension (C22)

From a vertical unit displacement field (u²²), the C₂₂ element of the homogenized elasticity tensor can be solved by

See FIG. 6B, which illustrates the deformed shape of horizontal extension homogenization of the unit cell.

$${C}_{22}^{H} = \frac{1}{Y} {\sum }_{e = 1}^{N} {( {u}_{e}^{22} )}^{T} {k}_{e} ( {u}_{e}^{22} ) \qquad ( 64 )$$

See FIG. 6B, which illustrates the deformed shape of horizontal extension homogenization of the unit cell.

TABLE 5
Deformed Shape of Vertical Extension
Homogenization of the Unit Cell.

| | | Peak | |
|---|---|---|---|
| | Number | Memory | |
| Solution | of | Usage | Solution |
| Method | Iterations | (MB) | (C₂₂) |
| Standard CG | 36 | 52.3 | 0.06 GPa |
| JFNK CG | 43 | 0.08 | 0.06 GPa |
| Adaptive | 96, 178 | 0.1 | 0.07 GPa |
| Dynamic | | | |
| Relaxation | | | |
| Abaqus | 10 | 67.1 | 0.06 GPa |
| Standard | | | |

3.3 Pure Shear (C12)

From a vertical and horizontal unit displacement field (u¹²), the C₁₂ element of the homogenized elasticity tensor can be solved by

$${C}_{12}^{H} = \frac{1}{Y} {\sum }_{e = 1}^{N} {( {u}_{e}^{12} )}^{T} {k}_{e} ( {u}_{e}^{12} ) \qquad ( 65 )$$

See FIG. 6B, which illustrates the deformed shape of pure shear homogenization of the unit cell.

TABLE 6
Performance of Standard CG, JFNK CG, aDR,
and Abaqus for Shear Homogenization

| | | Peak | |
|---|---|---|---|
| | Number | Memory | |
| Solution | of | Usage | Solution |
| Method | Iterations | (MB) | (C₁₂) |
| Standard CG | 53 | 52.8 | 0.158 GPa |
| JFNK CG | 64 | 0.08 | 0.158 GPa |
| Adaptive | 98, 123 | 0.1 | 0.169 GPa |
| Dynamic | | | |
| Relaxation | | | |
| Abaqus | 10 | 67.1 | 0.158 GPa |
| Standard | | | |

### Example 4: Hydrostatic Preloading Of An Acoustic Cloak

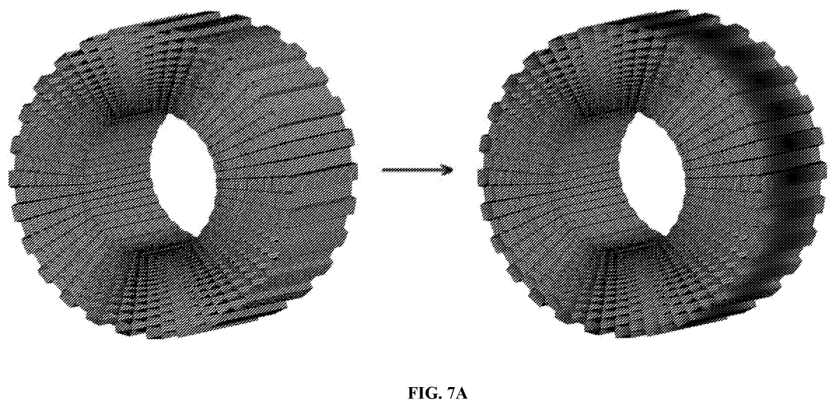

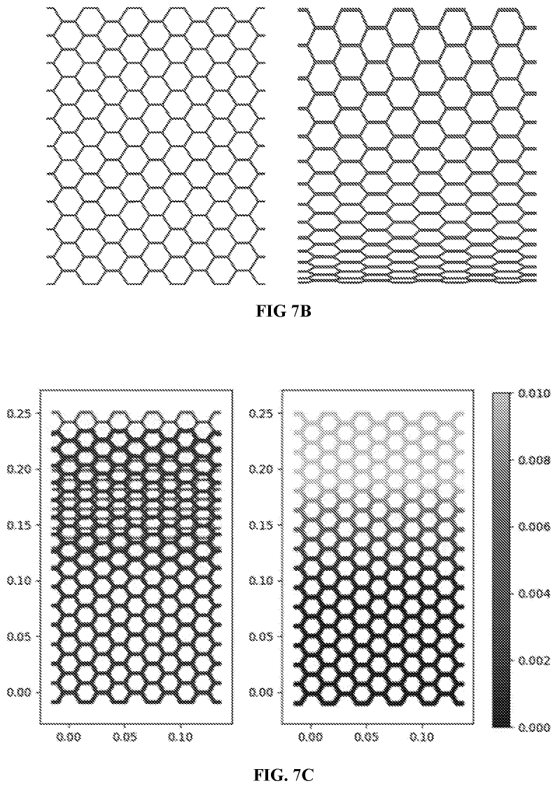

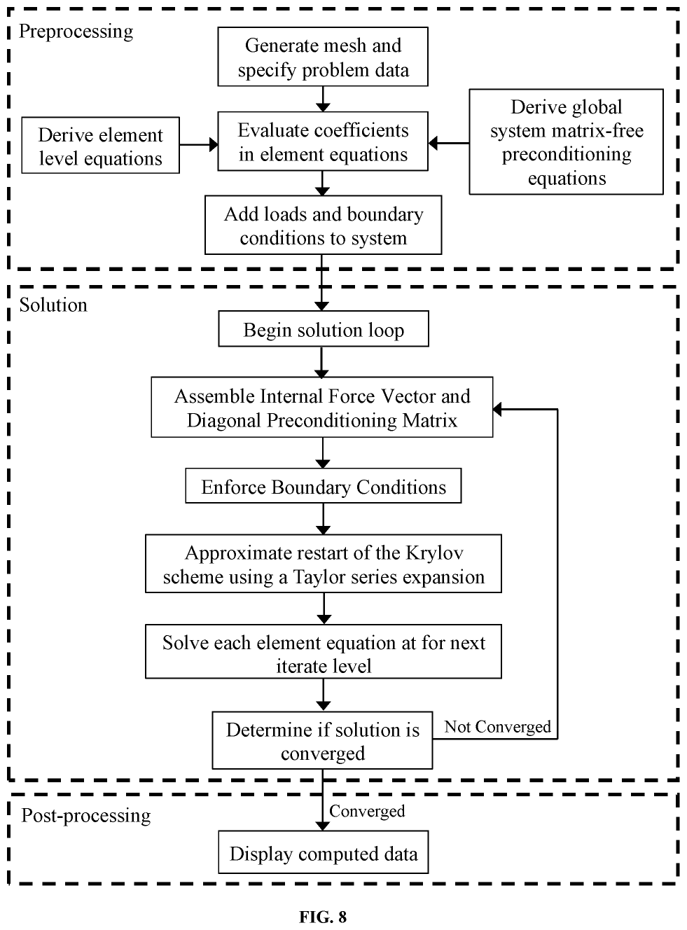

A functionally graded metamaterial structure was put under hydrostatic preload. The cloak was composed of 543,312 four-node, single-integration-point shell elements with hourglass control, as shown by FIG. 7. The mass and stiffness varied by unit cell and thus varied throughout the structure. The nominal material model properties were a linear elastic model with Young's modulus of 200 GPa, mass density of 7.83 g/cm3, a Poisson's ratio of 0.32, and shell thickness of 1 mm.

TABLE 7
Performance of Standard CG, JFNK CG, aDR,
and Abaqus for Hydrostatic Loading.

| | | Peak |
|---|---|---|
| | Number | Memory |
| Solution | of | Usage |
| Method | Iterations | (MB) |
| Standard CG | 2123 | 1857 |
| JFNK CG | 2573 | 18.2 |
| Adaptive | 100, 175 | 25.1 |
| Dynamic | | |
| Relaxation | | |
| Abaqus | 60 | 2055 |
| Standard | | |

### Example 5: Extensions to Homogenization of Non-Linear Materials: Hyperelasticity

The horizontal metamaterial unit cell extension described above was reanalyzed however with a Neo-Hookean material model. The homogenization process for a hyperelastic material is somewhat more involved than for linear elasticity (often strain energy must often be taken into account) however in an effort to simply show JFNK's advantages for homogenization of non-linear materials (or materials with complicated or non-existent tangent stiffness tensors), the process used was the same as the linear homogenization described above. A shear modulus of 1.5 MPa and bulk modulus of 10 GPa were used. Five load steps are used with the initial unit load doubled at each load step. In the case of dynamic relaxation, no load steps are taken because the analysis is dynamic—each time step can be thought of as a series of very small load steps. The number of Newton iterations used by Abaqus and standard CG is shown in Table 8 below. It is noted that JFNK does not require a fully derived, or even the existence, of a material tangent (Hales et al. 2012) making each iteration within each load step substantially easier to compute.

TABLE 8
Performance of Standard CG, JFNK CG, aDR,
and Abaqus for Hyperelastic Homogenization.

| | | | Peak |
|---|---|---|---|
| | | Number of | Memory |
| Solution | Number of | Iterations per | Usage |
| Method | Load Steps | Load Step | (MB) |
| Standard CG | 5 | 34, 41, 48, 52, 64 | 67.9 |
| JFNK CG | 5 | 41, 42, 51, 56, 71 | 0.21 |
| Adaptive | 1 | 94, 127 | 0.35 |
| Dynamic | | | |
| Relaxation | | | |
| Abaqus | 5 | 6, 4, 9, 4, 7 | 39.0 |
| Standard | | | |

As illustrated by the example above, the present disclosure is a memory efficient explicit algorithm for linear and nonlinear static equilibrium in the context of mechanical metamaterials and acoustic cloaking. The JFNK algorithm described is capable of handling geometric and material nonlinearities as well as all standard finite elements. This algorithm and the methods described herein may, for example, be applied to shells and elements with hourglass control in the context of mechanical metamaterial design. The JFNK method provides a useful alternative to dynamic relaxation for nonlinear static equilibrium computations using explicit finite element codes and standard matrix-based methods traditionally used in homogenization and preload calculations. Additionally, in the case of non-linear materials, JFNK requires no existence of the tangent stiffness which may not be known or could be very computationally expensive (e.g., polymers).

Although the algorithm is quite different than dynamic relaxation, it surprisingly requires no alteration of the explicit code architecture, making use of the same internal force function calls. This feature allows the incorporation of JFNK into an existing explicit dynamics code relatively noninvasive. Within the metamaterial design process, the prospect of only having a single code based on an explicit loop can be attractive since the cost of developing and maintaining two codes bases (static and explicit dynamics) can be substantial.

As indicated above, the algorithm's accuracy for homogenization of both linear and non-linear materials was verified against a well-known commercial finite element package, Abaqus. Although the average number of iterations needed was found to be higher in JFNK than standard preconditioned conjugate gradient, the memory efficiency was significantly improved. Additionally, specific problems were shown in which, due to memory requirements, both CG and Abaqus failed to find a solution where JFNK did. Furthermore, since the Abaqus matrix-based solution is not CG-based, care should be taken not to place too much weight on direct iterations comparisons to JFNK. As noted by Hales et al., comparisons between methods are difficult to assess. In the case of comparing JFNK to standard CG, the difference may ultimately come down to optimization of the operators—matrix multiplication in matrix-based CG and vectorization of the element loop in JFNK.

Although many JFNK variations exist, the present disclosure illustrates a subset which allow for minimal memory and algorithmic differences from dynamic relaxation; specifically, those which do not entail storage of many solution vectors and which do not require any matrix solutions. CG is used in this study, but it is noted that there are other potentially promising JFNK approaches, such as GMRES, that have still yet to be explored. Additionally, porting the algorithm to a heterogeneous computing platform provides an interesting avenue for future work. As computer architectures begins to favor cache sizes more similar to accelerators, a less memory intensive finite element formulation could prove to be valuable.

The present disclosure provides computers that are programmed or otherwise configured to implement methods of the disclosure. Such computer systems may comprise a central processing unit (CPU, also referred to as a “processor” herein), which can be a single core or multi-core processor, or a plurality of processors for parallel processing. The computer system also includes memory or memory location (e.g., random-access memory, read-only memory, flash memory), an electronic storage unit (e.g., hard disk), communication interface (e.g., network adapter) for communicating with one or more other systems, and peripheral devices, such as cache, other memory, data storage and/or electronic display adapters. The memory, storage unit, interface and peripheral devices may be in communication with the CPU through a communication bus (solid lines), such as a motherboard. The storage unit can be a data storage unit (or data repository) for storing data. The computer system can be operatively coupled to a computer network (“network”) with the aid of the communication interface. The network can be the Internet, an internet and/or extranet, or an intranet and/or extranet that is in communication with the Internet. The network in some cases is a telecommunication and/or data network. The network can include one or more computer servers, which can enable distributed computing, such as cloud computing. The network, in some cases with the aid of the computer system, can implement a peer-to-peer network, which may enable devices coupled to the computer system to behave as a client or a server. The CPU can execute a sequence of machine-readable instructions, which can be embodied in a program or software. The instructions may be stored in a memory location, such as the memory. The instructions can be directed to the CPU, which can subsequently program or otherwise configure the CPU to implement any of the methods and/or algorithms of the present disclosure.

In the interest of clarity, not all of the routine features are disclosed herein. It is understood that in the development of an actual implementation of the present disclosure, numerous implementation-specific decisions must be made in order to achieve specific goals, and that these specific goals will vary for different implementations. It will be appreciated that such efforts might be complex and time-consuming, but would nevertheless be a routine undertaking of engineering for a person of ordinary skill in the art, having the benefit of the present disclosure.

Furthermore, it is understood that the phraseology or terminology used herein is for the purpose of description and not of restriction, such that the terminology or phraseology of the present disclosure is to be interpreted in light of the teachings and guidance presented herein, in combination with knowledge available to a person of ordinary skill in the relevant art at the time of the invention. Moreover, it is not intended for any term in the specification or claims to be ascribed an uncommon or special meaning, unless explicitly set forth as such in the specification.

The various aspects disclosed herein encompass present and future known equivalents to the structural and functional elements referred to herein by way of illustration. Moreover, while various aspects and applications have been shown and described herein, it will be apparent to those of ordinary skill in the art having the benefit of this disclosure that many more modifications than those mentioned above are possible without departing from the inventive concepts disclosed herein. For example, one of ordinary skill in the art would readily appreciate that individual features from any of the exemplary aspects disclosed herein may be combined to generate additional aspects that are in accordance with the inventive concepts disclosed herein.

It is further understood that any combination of elements or steps described herein may be used alone or in combination with still further unrecited elements or steps. To that end, any reference to the transitional phrase “comprising” recited herein is expressly understood also to include support for alternative aspects directed to a closed set (i.e., “consisting of” only the recited elements) and for a semi-closed set (i.e., “consisting essentially of” the recited elements and any additional elements or steps that do not materially affect the basic and novel characteristics of the invention).

Although illustrative exemplary aspects have been shown and described, a wide range of modification, change and substitution is contemplated in the foregoing disclosure and in some instances, some features of the embodiments may be employed without a corresponding use of other features. Accordingly, it is appropriate that the appended claims be construed broadly and in a manner consistent with the scope of the embodiments disclosed herein.

1. A computer-implemented method for reducing the computational burden, in terms of time and resources, when modeling a problem involving shell finite elements, the method comprising:
a) a pre-processing phase, wherein a mesh is generated by a processor, and problem data is specified;
b) a solution phase comprising the steps of:
deriving one or more element equations;
deriving one or more global system matrix-free shell finite element equations, wherein said deriving step comprises defining elements that include i) bending and ii) membrane stiffness degrees of freedom;
executing, by the processor, calls to one or more internal force routines from an explicit dynamics element and material library to incorporate material models;
evaluating coefficients in said element equations using the derived element equations and the derived global system matrix-free shell finite element equations;
adding load and boundary conditions to said element equations; and
solving said element equations; and
c) a post-processing phase wherein computed data based on the solved element equations is displayed on a user interface of a display device.

2. The method of claim 1, wherein the step of deriving said global system matrix-free shell finite elements equations comprise equations for:
a) static homogenization,
b) functionally graded metamaterial design,
c) hydrostatic loading,
d) long-duration dynamics using implicit integration, and/or
e) vibroacoustics in the frequency domain.

3. The method of claim 1, wherein said step of deriving one or more global system matrix-free shell finite element equations further comprises one or more of the following steps:
a) defining a membrane elemental stiffness matrix;
b) defining a bending elemental stiffness matrix;
c) defining a preconditioning scheme for displacement degrees of freedom;
d) defining a preconditioning scheme for rotational degrees of freedom;
e) deriving associated algorithms and functions associated with said global-system matrix-free shell finite elements; and/or
f) computing a solution to said global-system matrix-free shell finite element equations.

4. The method of claim 1, wherein deriving one or more global system matrix-free shell finite element equations comprises one or more of the following steps:
a) selecting an iterative Krylov scheme;
b) defining an approximate restart of the iterative scheme using a Taylor series expansion;
c) determining an increment based on the tangent stiffness;
d) expressing an increment using only the action of the internal forces; and/or
e) using a selected Krylov scheme to solve for the next increment.

5. The method of claim 3, wherein the preconditioning scheme is defined as a diagonal array of elements of the membrane elemental stiffness matrix and the bending elemental stiffness matrix, respectively.

6. The method of claim 3, wherein said preconditioning scheme is defined using one or more unassembled shell element stiffness matrices.

7. A system for modeling a problem involving shell finite elements, comprising a processor configured to:
generate a mesh and receive user-specified problem data;
derive one or more element equations;
derive one or more global system matrix-free shell finite element equations, wherein said deriving step comprises defining elements that include i) bending and ii) membrane stiffness degrees of freedom;
execute calls to one or more internal force routines from an explicit dynamics element and material library to incorporate material models;
evaluate coefficients in said element equations using the derived element equations and the derived global system matrix-free shell finite element equations;
add load and boundary conditions to said element equations; and
solve said element equations.

8. The system of claim 7, wherein the processor is further configured to display computed data based on the solved element equations on a user interface of a display device.

9. The system of claim 7, wherein the processor is further configured to derive the global system matrix-free shell finite element equations by deriving equations for:
a) static homogenization,
b) functionally graded metamaterial design,
c) hydrostatic loading,
d) long-duration dynamics using implicit integration, and/or
e) vibroacoustics in the frequency domain.

10. The system of claim 7, wherein the processor is further configured to derive the one or more global system matrix-free shell finite element equations by:
a) defining a membrane elemental stiffness matrix;
b) defining a bending elemental stiffness matrix;
c) defining a preconditioning scheme for displacement degrees of freedom;
d) defining a preconditioning scheme for rotational degrees of freedom;
e) deriving associated algorithms and functions associated with said global-system matrix-free shell finite elements; and/or
f) computing a solution to said global-system matrix-free shell finite element equations.

11. The system of claim 7, wherein the processor is further configured to derive the one or more global system matrix-free shell finite element equations by:
a) selecting an iterative Krylov scheme;
b) defining an approximate restart of the iterative scheme using a Taylor series expansion;
c) determining an increment based on the tangent stiffness;
d) expressing an increment using only the action of the internal forces; and/or
e) using a selected Krylov scheme to solve for the next increment.

12. The system of claim 10, wherein the preconditioning scheme is defined as a diagonal array of elements of the membrane elemental stiffness matrix and the bending elemental stiffness matrix, respectively.

13. The system of claim 10, wherein the preconditioning scheme is defined using one or more unassembled shell element stiffness matrices.

14. A non-transitory computer readable medium storing executable instructions for modeling a problem involving shell finite elements, wherein said instructions include instructions that when executed will cause a processor to:
generate a mesh and receive user-specified problem data;
derive one or more element equations;
derive one or more global system matrix-free shell finite element equations, wherein said deriving step comprises defining elements that include i) bending and ii) membrane stiffness degrees of freedom;
execute calls to one or more internal force routines from an explicit dynamics element and material library to incorporate material models;
evaluate coefficients in said element equations using the derived element equations and the derived global system matrix-free shell finite element equations;
add load and boundary conditions to said element equations; and
solve said element equations.

15. The non-transitory computer readable medium of claim 14, further comprising instructions that when executed will cause a processor to:
display computed data based on the solved element equations on a user interface of a display device.
